(19) United States
(12) Patent Application Publication
(10) Pub. No.: US 2023/0103446 A1
(43) Pub. Date: Apr. 6, 2023

(54) MICROFLUIDIC NANOPORE SENSING DEVICES

(71) Applicant: Hewlett-Packard Development Company, L.P., Spring, TX (US)

(72) Inventor: Si-lam J. CHOY, Corvallis, OR (US)

(73) Assignee: Hewlett-Packard Development Company, L.P., Spring, TX (US)

(21) Appl. No.: 17/910,112

(22) PCT Filed: Apr. 10, 2020
(86) PCT No.: PCT/US2020/027631

§ 371 Date: Sep. 8, 2022

(51) Int. Cl. G01N 33/487; G01N 27/27; C12Q 1/6869; B01L 3/00

(52) U.S. Cl. CPC G01N 33/48721; B01L 3/5027; C12Q 1/6869; G01N 27/27; B01L 2300/0861

(57) ABSTRACT

The present disclosure is drawn to microfluidic nanopore sensing devices. The microfluidic nanopore sensing device can include a common electrolyte chamber; a discrete electrolyte chamber separated from the common electrolyte chamber by a nanopore opening therebetween; and an electrical circuit including multiple electrodes, where the common electrolyte chamber is electrically associated with a first electrode to provide a first polarity and the discrete electrolyte chamber is electrically associated with a second electrode to provide a second polarity that is opposite the first polarity. The device can also include an inlet channel fluidly coupled to the discrete electrolyte chamber via an inlet port and an outlet channel separated from the inlet channel that is also fluidly coupled to the discrete electrolyte chamber by an outlet port. In this example, the inlet channel can be fluidly coupled to a second discrete electrolyte chamber via a second inlet port.

### BACKGROUND

Microfluidic nanopore sensing devices can exploit chemical and physical properties of fluids on a microscale. These devices can be used for research, medical, and forensic applications, to name a few, to evaluate oranalyze fluids using very small quantities of sample and/or reagent to interact with the sample than would otherwise be used in a full-scale analysis device or system.

### BRIEF DESCRIPTION OF THE DRAWINGS

FIG. 1 graphically illustrates a schematic cross-sectional view of an example microfluidic nanopore sensing device in accordance with the present disclosure;

FIG. 2 graphically illustrates a schematic cross-sectional view of an example microfluidic nanopore sensing device arranged as a microfluidic array in accordance with the present disclosure;

FIG. 3A graphically illustrates a schematic bottom view of an example microfluidic nanopore sensing device in accordance with the present disclosure;

FIG. 3B graphically illustrates a schematic bottom view of an example microfluidic nanopore sensing device in accordance with the present disclosure;

FIG. 4 graphically illustrates a schematic microfluidic system in accordance with the present disclosure;

FIG. 5 is a flow diagram illustrating an example method of using a microfluidic nanopore sensing device in accordance with the present disclosure; and

FIG. 6 schematically illustrates a method of using a microfluidic nanopore sensing device and includes a cross-sectional view of a microfluidic nanopore sensing device in accordance with an example of the present disclosure.

### DETAILED DESCRIPTION

Microfluidic nanopore sensing devices can be used in a variety of applications, including biotechnology, drug screening, clinical diagnostic testing, etc. An example application of a microfluidic nanopore sensing device includes a nanopore sequencing device. Nanopore sequencing can be valuable in areas of microbiology, environmental research, microbiome, basic genome research, human genetics, diagnostic testing, cancer research, clinical research, plant research, transcriptome analysis, population scale genomics, animal research, and the like. Nanopore sequencing specifically allows for sequencing of a single biological sample without PCR amplification or chemical labeling. For example, during nanopore sequencing, a biological sample can be immersed in an electrolytic fluid on a first side of a nanopore, an electrical potential can be applied across the nanopore thereby generating a constant current through the nanopores and the biological sample can be transported through the nanopore from the first area to a second area. As the biological sample passes through the nanopore (or nanopores when there are multiple chambers), a change in the electrical properties can occur. This electrical change can correspond with a structure of the biological sample and can be correlated with the structure; thereby, indicating the structure of the biological sample passing therethrough. For example, nanopore sequencing can be used to determine a sequence of a nucleic acid. Each nucleotide base of the nucleic acid (adenine, quinine, cytosine, and thymine) has a known electrical potential and can interfere with the flow of ions through a nanopore. The change in electrical properties can be correlated to individual nucleotide bases or a group of bases that passes therethrough, and a sequence of the nucleic acid can be determined.

The present disclosure is drawn to microfluidic nanopore sensing devices, methods of manufacturing microfluidic nanopore sensing devices, and systems for conducting a biological assays. A microfluidic nanopore sensing device, for example, includes a common electrolyte chamber, a discrete electrolyte chamber, an electrical circuit, an inlet channel, and an outlet channel. The discrete electrolyte chamber is separated from the common electrolyte chamber by a nanopore opening therebetween. The electrical circuit includes multiple electrodes. The common electrolyte chamber is electrically associated with a first electrode to provide a first polarity and the discrete electrolyte chamber is electrically associated with a second electrode to provide a second polarity that is opposite the first polarity. The inlet channel is fluidly coupled to the discrete electrolyte chamber via an inlet port and the inlet channel is fluidly coupled to a second discrete electrolyte chamber via a second inlet port. The outlet channel is separated from the inlet channel and is fluidly coupled to the discrete electrolyte chamber by an outlet port. In one example, the outlet channel can be fluidly coupled to the second discrete electrolyte chamber via a second outlet port. In another example, the nanopore opening can be defined by an inorganic membrane. In another example, the common electrolyte chamber can further include an electrolyte inlet and an electrolyte outlet. In a further example, the microfluidic nanopore sensing device can include a microfluidic array with a series of discrete electrolyte chambers individually separated from the common electrolyte chamber by individual nanopore openings. The discrete electrolyte chambers can individually include a corresponding series of second electrodes to provide the second polarity. In another example, the individual discrete electrolyte chambers can be independently fluidly coupled to its own input channel fluidly coupled to a corresponding inlet port, and its own outlet channel fluidly coupled to a corresponding outlet port. In yet another example, the individual discrete electrolyte chambers can be assembled in parallel so that the inlet channel can be fluidly coupled to an inlet port of the individual discrete electrolyte chamber and the outlet channel can be fluidly coupled to an outlet port of the individual discrete electrolyte chamber. In a further example, the microfluidic nanopore sensing device can further include a first loading opening to load electrolytic fluid into the common electrolyte chamber and a second loading opening to load electrolytic fluid into the discrete electrolyte chamber through the inlet channel and the inlet port.

In another example, a microfluidic system includes, for example, a microfluidic nanopore sensing device and a non-polar fluid. The microfluidic nanopore sensing device can include a common electrolyte chamber, a discrete electrolyte chamber, an electrical circuit, an inlet channel, and an outlet channel. The discrete electrolyte chamber can be separated from the common electrolyte chamber by a nanopore opening therebetween. The electrical circuit can include multiple electrodes. The common electrode chamber can be electrically associated with a first electrode to provide a first polarity. The discrete electrolyte chamber can be electrically associated with a second electrode to provide a second polarity that can be opposite the first polarity. The inlet channel can be fluidly coupled to the discrete electrolyte chamber via an inlet port, and the inlet channel can be fluidly coupled to a second discrete electrolyte chamber via a second inlet port. The outlet channel can be separated from the inlet channel and can be fluidly coupled to the discrete electrolyte chamber by an outlet port. The non-polar fluid can be contained within or loadable within the inlet channel, the outlet channel, or both. In one example, the system can further include an electrolytic fluid selected from potassium chloride, silver chloride, sodium chloride, lithium chloride, magnesium chloride, calcium chloride, potassium phosphate, sodium phosphate, lithium phosphate, magnesium phosphate, calcium phosphate, potassium carbonate, calcium carbonate, sodium carbonate, lithium chloride, magnesium carbonate, sulfuric acid, potassium hydroxide, or a combination thereof.

A method of using a microfluidic nanopore sensing device, in another example, includes loading a sample electrolytic fluid including an electrolytic fluid and a biological sample into a common electrolyte chamber, and loading an electrolytic fluid into a discrete electrolyte chamber, where the discrete electrolyte chamber can be separated from the common electrolyte chamber by a nanopore opening therebetween. Loading into the discrete electrolyte chamber can occur by passing the electrolytic fluid through an inlet channel and into the discrete electrolyte chamber via an inlet port. The method further includes flushing the inlet channel with air, a non-polar fluid or sequentially air and then non-polar fluid, and venting the electrolytic fluid from the discrete electrolyte chamber through the outlet port and into an outlet channel. In one example, the nanopore separating the discrete electrolyte chamber from the common electrolyte chamber can be formed after loading the sample electrolytic fluid, after loading the electrolytic fluid, and after flushing the inlet channel with the air, the non-polar fluid, or the combination thereof. In another example, the method can further include loading a second discrete electrolyte chamber with an electrolytic fluid. In another example, the biological sample can be a nucleic acid, and the nanopore opening can have a diameter from 0.5 nm to 2.5 nm. In this example, the method can further include sequencing the nucleic acid. In a further example, the microfluidic nanopore sensing device can further include an electrical circuit including multiple electrodes. The common electrode chamber can be fluidly coupled to a first electrode to provide a first polarity and the discrete electrolyte chamber can be fluidly coupled to a second electrode to provide a second polarity that can be opposite the first polarity.

It is noted that when discussing the microfluidic nanopore sensing devices, the microfluidic methods, or the microfluidic systems, such discussions of one example are to be considered applicable to the other examples, whether or not they are explicitly discussed in the context of that example. Thus, in discussing an inlet channel in the context of the microfluidic nanopore sensing devices, such disclosure is also relevant to and directly supported in the context of the microfluidic systems and the methods of using a microfluidic nanopore sensing device, and vice versa.

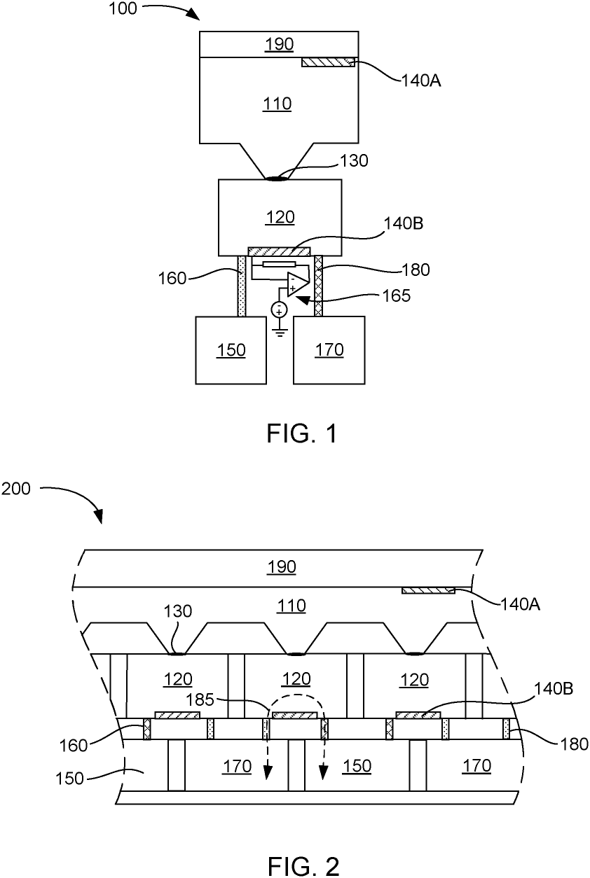

Turning now to the figures for further detail, as an initial matter, there are several components of the microfluidic nanopore sensing devices shown that are common to multiple examples, and thus, the common reference numerals are used to describe various features. Thus, a general description of a feature in the context of a specific figure can be relevant to the other example figures shown, and as a result, individual components need not be described and then re-described in context of another figure. In the following example descriptions, FIGS. 1-4 and 6 can be considered simultaneously in the description of the figures to the extent relevant by common reference numerals, for example.

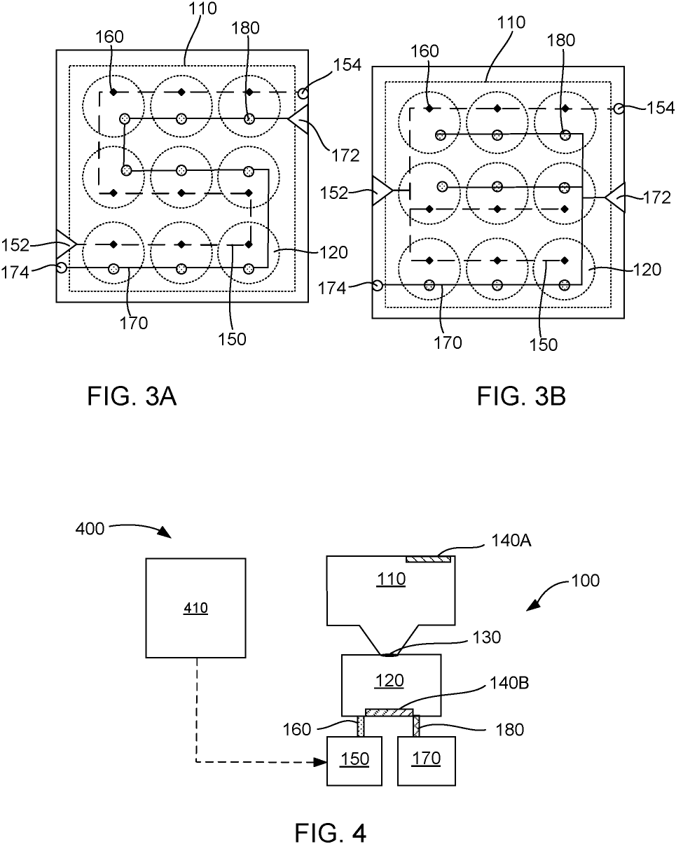

With more specific reference to FIG. 1, a schematic cross-sectional view of an example microfluidic nanopore sensing device 100 is shown. The microfluidic nanopore sensing device can include a common electrolyte chamber 110, a discrete electrolyte chamber 120 separated from the common electrolyte chamber by a nanopore opening 130. The microfluidic nanopore sensing device can also include electrical circuitry including multiple electrodes (140A and 140B) where the common electrode chamber is electrically associated with a first electrode 140A to provide a first polarity and the discrete electrolyte chamber is electrically associated with a second electrode 140B to provide a second polarity that is opposite the first polarity. The electrical circuitry may also include, for example, electrical sensor components 165 associated with one or both of the multiple electrodes. The electrical sensor component(s) shown by way of example in this FIG. may also be present in subsequent FIGS., even though not specifically shown. For example, it is understood that the electrical sensor of this configuration or any other sensor configuration can be present and electrically associated with any of the first or second electrodes shown herein. They are not shown in those FIGS due to figure space constraint. The microfluidic nanopore sensing device can also include inlet channel 150 fluidly coupled to the discrete electrolyte chamber via an inlet port 160 and an outlet channel 170 fluidly coupled to the discrete electrolyte chamber by an outlet port 180. The ports are shown as elongated ports in this and other examples, but could be, for example, simply an opening that connects the inlet and/or outlet channels to its respective discrete electrolyte chamber(s). The cross-sectional view shown in FIG. 1 does not illustrate the inlet channel fluidly coupling to a second discrete electrolyte chamber via a second inlet port, as the view shown is a height by width cross-sectional view. However, FIGS. 3A and 3B illustrate an alternative view which graphically illustrates how the inlet channel and the outlet channel can be fluidly coupled to multiple discrete electrolyte chambers via inlet ports and/or outlet ports. Thus, in further detail, as the inlet channel is shown in cross-section, the length of the channel is not apparent in FIG. 1 (as the length would traverse into and out of the page). Thus, with this understanding, in this example, the inlet port can channel fluid between the inlet channel and the discrete electrolyte chamber through a side wall of the inlet channel. Likewise, as shown in FIG. 1, the outlet port can channel fluid between the out channel and the discrete electrolyte chamber through a side wall of the outlet channel. Thus, if there are multiple discrete electrolyte chambers positioned in series along the inlet and/or out channels, respective ports can connect these channels to multiple discreet electrolyte chambers through their side walls along a length of the respective channels.

In another example, as illustrated in a cross-sectional view in FIG. 2, the microfluidic nanopore sensing device can be arranged as a microfluidic array 200. The microfluidic array can include a common electrolyte chamber 110, a discrete electrolyte chamber 120 separated from the common electrolyte chamber by a nanopore opening 130, an electrical circuit including multiple electrodes (140A and 140B) where the common electrode chamber is electrically associated with a first electrode 140A to provide a first polarity and the discrete electrolyte chamber is electrically associated with a second electrode 140B to provide a second polarity that is opposite the first polarity, an inlet channel 150 fluidly coupled to the discrete electrolyte chamber via an inlet port 160, and an outlet channel 170 fluidly coupled to the discrete electrolyte chamber by an outlet port 180. In FIG. 2 the common electrolyte chamber is paired with multiple discrete electrolyte chambers. The discrete electrolyte chambers each include a second electrode positioned therein. Further, the inlet channel may include an inlet port for multiple discrete electrolyte chambers and each outlet channel may include an outlet port for multiple discrete electrolyte chambers. For example, the inlet channel can include a first inlet port fluidly coupled to a first discrete electrolyte chamber, a second inlet port fluidly coupled to a second discrete electrolyte chamber, a third inlet port fluidly coupled to a third discrete electrolyte chamber, and so forth. The outlet channel can include a first outlet port fluidly coupled to a first discrete electrolyte chamber, a second outlet port fluidly coupled to a second discrete electrolyte chamber, a third outlet port fluidly coupled to a third discrete electrolyte chamber, and so forth. The number of inlet ports and//or outlet ports that may be correspondingly connected to different discrete electrolyte chambers is not limited. An example flow path 185 is shown that can be bi-directional or unidirectional. In this example, three can be inlet ports and outlet ports that share a channel so that the channel acts as an inlet channel for an inlet port and also can act as an outlet channel for an outlet port. In some examples, inlet channels can likewise be fluidly coupled together, and outlet channels can likewise be fluidly coupled together. These arrangements are not shown in this example, but are shown in an alternative view in FIGS. 3A and 3B, for example. The discrete electrolyte chambers can be loaded, evacuated, vented, filled, or the like, along this flow path, for example, using electrolyte fluid, air, oil, or other fluids, as described in further detail hereinafter. The inlet channel and the outlet channel do not directly connect to the discrete electrolyte chamber. These channels may be fluidly coupled to discrete electrolyte chambers via inlet port(s) and outlet port(s).

In some examples, the common electrolyte chamber 110 can be its own stand-alone chamber. In other examples, the common electrolyte chamber can be a multiple part chamber connected by microfluidics or fluidic channels. That stated the microfluidic channel in such an arrangement can connect multiple chamber portions larger than the size of the nanopore opening that separates the common electrolyte chamber from the discrete electrolyte chamber (or chambers). In further detail, the common electrolyte chamber can be enclosed by a lid. The lid can be any configuration suitable for forming or covering the common electrolyte chamber.

The microfluidic nanopore sensing device, in further detail, can include a common electrolyte chamber. The common electrolyte chamber is not particularly limited in size, dimension, or shape, but in one example, the common electrolyte chamber can have a volume from about 50 µm to about 2,000 µm, from about 500 µm to about 1,500 µm, or from about 50 µm to about 750 µm, for example. The common electrolyte chamber can, for example, have an interior in a shape of a cube, cuboid (a.k.a. rectangular prism), cone, cylinder, triangular prism, polygonal prism, triangular based pyramid, square-based pyramid, polygonal based pyramid, spherical, hemi-spherical, or the like. In one example, the common electrolyte chamber can have an interior in a shape of a cube or cuboid.

The common electrolyte chamber, in some examples, can further include an electrolyte inlet and an electrolyte outlet. The electrolyte inlet and the electrolyte outlet can be sized and shaped to permit loading of a sample electrolytic fluid into the common electrolyte chamber. In some examples, the electrolyte inlet and/or the electrolyte outlet can be an opening. The opening may or may not be enclosable with a seal. In yet other examples the electrolyte inlet and the electrolyte outlet can include a self-sealing septum. The self-sealing septum can be penetrated via a needle. In some examples, the electrolyte inlet and the electrolyte outlet can be located in the lid and can be fluidly connected to the common electrolyte chamber. In some examples, the common electrolyte chamber can be fluidly coupled to other ports, such as vents or other structures for facilitating fluid flow to or through the common electrolyte chamber.

The common electrolyte chamber can house a first electrode 140A. The first electrode can provide a positive or a negative charge. A charge from the first electrode can flow to an electrolytic fluid that can be housed within the common electrolyte chamber and can thereby carry a charge throughout the common electrolyte chamber.

A common electrolyte chamber can be separated from and fluidly coupled to a discrete electrolyte chamber. In some examples, a common electrolyte chamber can be individually and fluidly coupled to an individual discrete electrolyte chamber as shown in FIG. 1. In other examples a common electrolyte chamber can be fluidly coupled to multiple discrete electrolyte chambers as shown in FIG. 2. A quantity of discrete electrolyte chambers that can be fluidly coupled with the common electrolyte chamber is not particularly limited. In some examples, the common electrolyte chamber can be fluidly coupled with from 1 to 10,000, from 2 to 5,000, from 10 to 10,000, from 20 to 10,000, from 50 to 5,000, or from 500 to 5,000 discrete electrolyte chambers.

The discrete electrolyte chamber is not particularly limited in size. Example volumes, for example, for individual discrete electrolyte chambers can be from 0.1 nL to 50 nL, from 0.1 nL to 20 nL, from 0.1 nL to 10 nL, from 0.1 to5 nL, from 0.5 nL to 3 nL, or from 0.1 nL to 1 nL. Dimensions, for example, may be based on length x width x height, and can be, for example, from 20 µm x 20 µm x 20 µm to 500 µm x 500 µm x 500 µm, though these dimensions are not intended to be limiting. The discrete electrolyte chamber is also not particularly limited in shape. For example, the discrete electrolyte chamber can have an interior that can be in the shape of a cube, cuboid (a.k.a. rectangular prism), cone, cylinder, triangular prism, polygonal prism, triangular based pyramid, square-based pyramid, polygonal based pyramid, spherical, hemi-spherical, or the like. In one example, the discrete electrolyte chamber can be in the shape of a cube or cuboid. In another example, the discrete electrolyte chamber can have an interior that can be in the shape of a cuboid elongated channel. In yet another example, the discrete electrolyte chamber can have an interior that can be in the shape of a cylinder.

In some examples, the discrete electrolyte chamber can be recessed in a substrate. Thus, the substrate can include (or define in full or in part) multiple discrete electrolyte chambers. A variety of substrate materials can be used. For example, the substrate can include a material selected from glass, quartz, polyamide, polydimethylsiloxane, silicon, SU8, polystyrene, polycarbonate, polymethyl methacrylate, polyethylene, poly(ethylene glycol) diacrylate, polypropylene, perfluoroalkoxy, fluorinated ethylene propylene, polyurethane, cyclic olefin polymer, cyclic olefin copolymer, phenolics, or a combination thereof. In one example, the substrate can include polydimethylsiloxane. In another example, the substrate can include polycarbonate. In yet another example, the substrate can include silicon. The substrate may be a single monolithic material, or may be a composited layered material of one material or multiple materials, for example.

The discrete electrolyte chamber can house a second electrode 140B. The second electrode can provide a positive or a negative charge that can be opposite a charge provided by the first electrode that is present in the common electrolyte chamber. A charge of the second electrode can flow through an electrolytic fluid that can be housed within the discrete electrolyte chamber; thereby carrying a charge throughout the discrete electrolyte chamber that can be opposite a charge in the common electrolyte chamber. Opposite charges from the first electrode and the second electrode can create a polarity in the microfluidic nanopore sensing device. The first electrode can provide a first polarity while the second electrode can provide a second polarity.

The first electrode of the common electrolyte chamber and the second electrode of the discrete electrolyte chamber when considered in combination can form an electrical circuit. In some examples, the electrical circuit can include multiple second electrodes that are individually present in a corresponding individual discrete electrolyte chamber, in the common chamber, or a combination thereof. In one example, the multiple electrodes of the electrical circuit can include multiple electrodes housed within individual discrete electrolyte chambers of the microfluidic nanopore sensing device so that the electrode can be put in electrical communication of fluid when introduced into the discrete electrolyte chamber(s). Furthermore, the common electrolyte chamber can include a first electrode that can be in electrical communication with the multiple electrodes (of opposite charge) independently present in the individual discrete electrolyte chambers. Thus, several discrete electrolyte chambers can include an individual second electrode, and the electrical circuit can include a combination of these electrodes working together as the presence of the various fluids may partially dictate. In another example, multiple electrodes in the common chamber can be used to balance an impedance of the second electrodes present in individual discrete electrolyte chambers.

The common electrolyte chamber can be separated from a discrete electrolyte chamber by a shared dielectric wall with a nanopore opening therebetween. The shared dielectric wall can be a monolithic material, or can be a layered or composited material of one or more layer types. The shared dielectric wall can prevent an electric charge from flowing therethrough and can be used to maintain a charge of the common electrolyte chamber separate from a charge of the discrete electrolyte chamber. The shared dielectric wall can include for example, a material selected from semiconductors, polymers, oxides, ceramics, or the like. In one example, a dielectric wall material can include hafnium oxide, silicon dioxide, silicon nitride, SU8, or a combination thereof. In one example, the shared dielectric wall can include SU8. In another example, the dielectric wall material can include hafnium oxide. A thickness of the shared dielectric wall may be very thin, e.g., as thin as possible or practical, to differentiate between nucleotides, but may also be thick enough to insulate a charge in the common electrolyte chamber from a charge in the discrete electrolyte chamber. Accordingly, a thickness of the shared dielectric wall can vary. In an example, the shared dielectric wall can be thinner near the nanopore opening and thicker in portions away from the nanopore opening.. A wall material having a higher dielectric constant can be a better insulator than a wall material having a lower dielectric constant, when considered with respect to one another. Therefore, a wall material with a higher dielectric constant can be a thinner layer and can provide the same insulation as a thicker layer of a wall material with a lower dielectric constant. In some examples, the shared dielectric wall can have a thickness from 1 nm to 10 nm, from 2 nm to 8 nm, or from 1 nm to 5 nm.

The nanopore opening separating the common electrolyte chamber from the discrete electrolyte chamber can include an opening defined by an organic membrane or an inorganic membrane. As used herein, an “organic membrane” can refer to a nanopore opening formed from pore-forming protein molecules which can be suspended in a lipid membrane. Examples of pore-forming protein molecules can include alpha hemolysin, aerolysin, MSpa porin, and the like. A diameter of the opening of an organic nanopore can be determined by the protein molecule making up the nanopore. An “inorganic membrane” can refer to a solid-state nanopore formed in a membrane of an inorganic material. For example, a solid-state nanopore can be formed in silicon, silicon nitride, glass, graphene, elastomeric materials, polymeric materials, or the like. In some examples solid-state nanopores can be formed by laser-assisted pulling of a glass capillary, ion-beam sculpting, electron beam sculpting, dielectric breakdown, wet etching, electrochemical anodization, metal assisted chemical etching, metal deposition, atomic layer deposition, or nano-imprint lithography. A diameter of an opening of an inorganic nanopore can be less limited by a material composition of the nanopore than an organic nanopore. The diameter of the nanopore can be determined by the limits of the manufacturing method and the desired application of the manufacturer.

The nanopore opening can vary in diameter size depending on application of the microfluidic nanopore sensing device. The nanopore opening may be sized to permit a biological sample to pass therethrough without folding upon itself. As used herein, a “biological sample” refers to a molecule from a living organism. In some examples, a biological sample can include ssDNA, dsDNA, ssRNA, dsRNA, peptide nucleic acid complexes, RNA ligand complexes, polynucleotides, nucleosides, protein molecules, exosomes, viruses, or the like. By way of example, a nanopore opening for sequencing a nucleic acid can have a diameter from 0.5 nm to 2.5 nm; while a nanopore opening for sequencing a large virus can have a diameter from 20 nm to 150 nm. With this in mind, the nanopore opening of the microfluidic nanopore sensing device can have a diameter from 0.5 nm to 150 nm. In yet other examples, the nanopore opening can have a diameter from 0.5 nm to 5 nm, from 0.5 nm to 2.5 nm, from 0.5 nm to 1.5 nm, from 0.5 nm to 50 nm, from 1 nm to 25 nm, from 25 nm to 75 nm, from 20 nm to 80 nm, or from 50 nm to 150 nm.

The microfluidic nanopore sensing device can further include an inlet channel that can be fluidly coupled to the discrete electrolyte chamber by an inlet port. The inlet channel can be configured to permit loading of an electrolytic fluid, air, or a non-polar fluid to the discrete electrolyte chamber. The microfluidic nanopore sensing device can also include an outlet channel that can be separated from the inlet channel and coupled to the discrete electrolyte chamber by an outlet port. The outlet channel can be configured to permit flushing of an electrolytic fluid, air, or a non-polar fluid from the discrete electrolyte chamber.

The inlet channel and the outlet channel can be used to provide fluid to (via the inlet port) and pass fluid from (via the outlet port) the discrete electrolyte chamber. In some examples, the inlet channel and the outlet channel do not pass fluid to the common electrolyte chamber. It is noted that the terms “inlet” and “outlet” do not infer that these elements (inlet channel, outlet channel, inlet port, or outlet port) interact with the discrete electrolyte chamber in one direction, though that could be the case. In some instances, there may be occasion for the fluid to flow “backwards” or “bi-directionally,” and thus the terms “inlet” and “outlet” can be used because at some point during operation, these elements act as inflow of fluid and outflow of fluid, respectively, relative to the discrete electrolyte chamber. Accordingly a structure of an inlet channel may serve as an inlet or an outlet and a structure of an outlet channel may serve as an outlet or an inlet.

A configuration of the inlet channel and the outlet channel are not particularly limited. The cross-sectional dimension of these channels can be circular, oval, rectangular, square, or of any other desired or convenient dimension for a particular application. In an example, these channels can be linear channels. In some examples, the channels can be similarly sized and shaped. The inlet channel and the outlet channel can individually have a channel width, at the shortest location along the x-axis, from 20 µm to 500 µm, from 50 µm to 250 µm, from 20 µm to 120 µm, from 150 µm to 450 µm, from 100 µm to 500 µm, or from 40 µm to 200 µm. The inlet channel and the outlet channel can individually have a channel height, at the shortest location along the y-axis, from 20 µm to 1,000 µm, from 100 µm to 500 µm, from 20 µm to 80 µm, from 250 µm to 750 µm, from 500 µm to 1,000 µm, or from 20 µm to 400 µm. A length along the z-axis of the inlet channel, the outlet channel, or a combination thereof can vary based on an arrangement of the microfluidic nanopore sensing device.

In some examples, the inlet channel and outlet channel can be arranged to fluidly couple with multiple discrete electrolyte chambers. The fluid coupling can occur via multiple inlet ports and outlet ports that can be located in a sidewall of the inlet channel or the outlet channel. Individual inlet ports and individual outlet ports can be coupled to individual discrete electrolyte chamber. In some examples, the inlet channel may be fluidly coupled to a second discrete electrolyte chamber via a second inlet port. The outlet channel may be fluidly coupled to the discrete electrolyte chamber by an outlet port. The number of inlet ports and outlet ports is not particularly limited, these ports may be used to fluidly couple the inlet channel or the outlet channel with individual discrete electrolyte chambers. The inlet channel and the outlet channel may not directly couple to the discrete electrolyte channel. Some example alternative configurations of the inlet channel 150 and the outlet channel 170 are illustrated in FIGS. 3A and 3B. These figures schematically depict a bottom side view of a microfluidic nanopore sensing device 100 including a common electrolyte chamber 110, several discrete electrolyte chambers 120, inlet ports 160, outlet ports 180, channel access openings 152 and 172, and waste outlets 154 and 174. FIG. 3A schematically illustrates an example serpentine arrangement of the inlet channel and the outlet channel. FIG. 3B schematically illustrates an example parallel arrangement of the inlet channel and the outlet channel.

A wall material of the inlet channel, the outlet channel, or a combination thereof can include any material operable to separate the channels from one another. However, in some examples, the wall material of the inlet channel, the outlet channel, or a combination thereof can include a dielectric material. For example, a dielectric material can include semiconductors, polymers, oxides, ceramics, or the like. In an example, the wall material can be independently selected from SU8, silicon, silicon dioxide, silicon nitride, polydimethylsiloxane, or a combination thereof.

In some examples, the microfluidic nanopore sensing device can further include a loading opening to load electrolytic fluid into the discrete electrolyte chamber through the inlet channel and inlet port, a loading opening to load air or a non-polar fluid into the discrete electrolyte chamber and/or flush an electrolytic fluid from the discrete electrolyte chamber, or a combination thereof. The channel access openings can work in combination with the waste outlets that can be opened to release air, electrolytic fluid, and/or a non-polar fluid from the inlet channel and/or the outlet channel.

In yet other examples, the microfluidic nanopore sensing device can further include integrated electrical elements that can be positioned to interact with an electrolytic fluid when an electrolytic fluid is located in the microfluidic nanopore sensing device. In some examples, the integrated electrical elements can include circuitry, resistors, transistors, capacitors, inductors, diodes, light emitting diodes, transistors, converters, conductive wires, conductive traces, photosensitive components, thermal sensitive components, semiconductor, and the like. In an example, the microfluidic nanopore sensing device can further include a resistor and the resistor can be a FET controlled resistor positioned in the dielectric chamber. In some examples an FET controlled resistor can enable inertial pumping that can be operable to assist in filling the dielectric chamber with an electrolytic fluid. The integrated electrical components can be in electrical communication with circuity or other components inside or outside of the microfluidic nanopore sensing device via a wire, a trace, a network of wires, a network of traces, an electrode, a conductive pad, and/or any other electrical communication structure that may or may not be embedded in the microfluidic nanopore sensing device. In one example, the microfluidic nanopore sensing device can further include integrated electrical elements that can be positioned in the discrete electrolyte chamber.

In some examples, the microfluidic nanopore sensing device can be configured as a microfluidic array. The microfluidic array can include a series of discrete electrolyte chambers individually separated from the common electrolyte chamber by individual nanopore openings. The discrete electrolyte chambers can be arranged in parallel or in series. In an example, the discrete electrolyte chamber can be arranged in parallel. The discrete electrolyte chambers in the array can individually include second electrodes to provide the second polarity. The individual discrete electrolyte chambers of the array can be fluidly coupled with individual common electrolyte chambers or the array can fluidly couple multiple discrete electrolyte chambers with a single common electrolyte chamber. In some examples, an individual inlet channel can include several inlet ports that can be fluidly coupled to several discrete electrolyte chambers. For example there can be an inlet port for each discrete electrolyte chamber in the microfluidic array.

In yet other examples, individual discrete electrolyte chambers can be independently fluidly coupled to their own input channel fluidly coupled thereto via a corresponding inlet port, and their own outlet channel fluidly coupled thereto via a corresponding outlet port. In some examples, individual discrete electrolyte chambers can be assembled in parallel so that the inlet channel can be fluidly coupled to an inlet port of the individual discrete electrolyte chamber and the outlet channel can be fluidly coupled to an outlet port of the individual discrete electrolyte chamber.

Further presented herein is a microfluidic system. An example microfluidic system 400 can include a microfluidic nanopore sensing device 100 and a non-polar fluid 410 as illustrated in FIG. 4. The microfluidic nanopore sensing device can include a common electrolyte chamber 110, a discrete electrolyte chamber 120 separated from the common electrolyte chamber by a nanopore opening 130, an electrical circuit including multiple electrodes (140A and 140B) where the common electrolyte chamber can be electrically associated with a first electrode 140A to provide a first polarity and the discrete electrolyte chamber can be electrically associated with a second electrode 140B to provide a second polarity that can be opposite the first polarity, an inlet channel 150 fluidly coupled to the discrete electrolyte chamber via an inlet port 160 and an outlet channel 170 fluidly coupled to the discrete electrolyte chamber by an outlet port 180. The non-polar fluid can be contained within or can be loadable within the inlet channel, the outlet channel, or both.

In further examples, the microfluidic nanopore sensing device can be as described above. The microfluidic nanopore sensing device can be a microfluidic array. The non-polar fluid, in further detail, can be selected from an oil, silicone oil, isoparaffin, liquid hydrocarbon, toluene, or a combination thereof. In one example, the non-polar fluid can be an oil. In some examples, the non-polar fluid can be packaged in a separate container from the microfluidic nanopore sensing device and may be loaded within the microfluidic nanopore sensing device prior to use.

In an example, the system can further include an electrolytic fluid. The electrolytic fluid can be contained within or can be loadable within the common electrolyte chamber, the discrete electrolyte chamber, or both. In some examples, the electrolytic fluid can be packaged in a separate container from the microfluidic nanopore sensing device and may be loaded within the microfluidic nanopore sensing device prior to use.

The electrolytic fluid can be selected from potassium chloride, silver chloride, sodium chloride, lithium chloride, magnesium chloride, calcium chloride, potassium phosphate, sodium phosphate, lithium phosphate, magnesium phosphate, calcium phosphate, potassium carbonate, calcium carbonate, sodium carbonate, lithium chloride, magnesium carbonate, sulfuric acid, potassium hydroxide, or a combination thereof. In an example, the electrolytic fluid can be selected from potassium chloride, silver chloride, sodium chloride, or magnesium chloride. In another example, the electrolytic fluid can be potassium chloride.

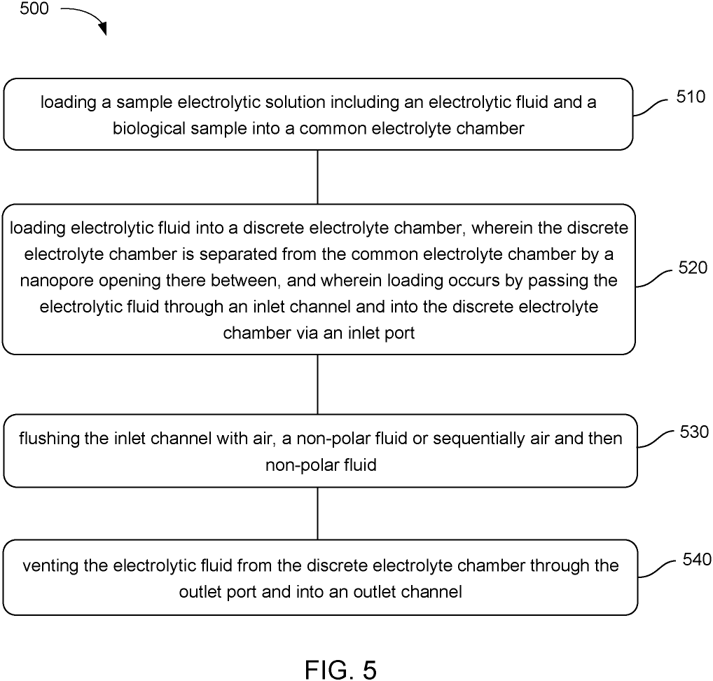

Further presented herein is a method 500 of using a microfluidic nanopore sensing device, as shown in FIG. 5. The method can include loading 510 a sample electrolytic fluid including an electrolytic fluid and a biological sample into a common electrolyte chamber and loading 520 electrolytic fluid into a discrete electrolyte chamber, where the discrete electrolyte chamber can be separated from the common electrolyte chamber by a nanopore opening therebetween. The loading can occur by passing the electrolytic fluid through an inlet channel and into the discrete electrolyte chamber via an inlet port. In some examples, the outlet port and the outlet channel can create a natural capillary break that may prevent or reduce a flow of the electrolytic fluid out of the discrete electrolyte chamber. In another example, the loading can occur while blocking electrolytic fluid egress out of an outlet port while allowing air to pass therethrough. The loading of the common electrolyte chamber and the discrete electrolyte chamber can occur in any order. For example, the discrete electrolyte chamber can be loaded prior to loading the common electrolyte chamber. In another example, the common electrolyte chamber can be loaded prior to loading the discrete electrolyte chamber. The method can further include flushing 530 the inlet channel with air, a non-polar fluid, or sequentially air and then non-polar fluid and venting 540 the electrolytic fluid from the discrete electrolyte chamber through the outlet port and into an outlet channel. In some examples, flushing of the inlet channel with air, a non-polar fluid, or air then the non-polar fluid can occur after nanopore sequencing.

In yet another example, the microfluidic nanopore sensing device can further include an electrical circuit including multiple electrodes. The common electrolyte chamber can be fluidly coupled to a first electrode to provide a first polarity and the discrete electrolyte chamber can be fluidly coupled to a second electrode to provide a second polarity that can be opposite the first polarity. In a further example, the microfluidic nanopore sensing device can be as described above.

In one example, the nanopore opening separating the discrete electrolyte chamber from the common electrolyte chamber can be formed after loading the sample electrolytic fluid and the electrolytic fluid, and after flushing the inlet channel with the air, the non-polar fluid, or a combination thereof. The formation of the nanopore opening can occur, in one example, via dielectric breakdown. For example, a voltage difference can be applied due to a polarity between the first electrode and the second electrode. In one example, the voltage difference applied thereto can range from 0.05 V to 15 V, from 0.1 V to 10 V, from 5 V to 10 V, or from 2 V to 8 V. The voltage applied can result in the formation of the nanopore opening due to a dielectric breakdown of a shared dielectric wall between the common electrolyte chamber and the discrete electrolyte chamber.

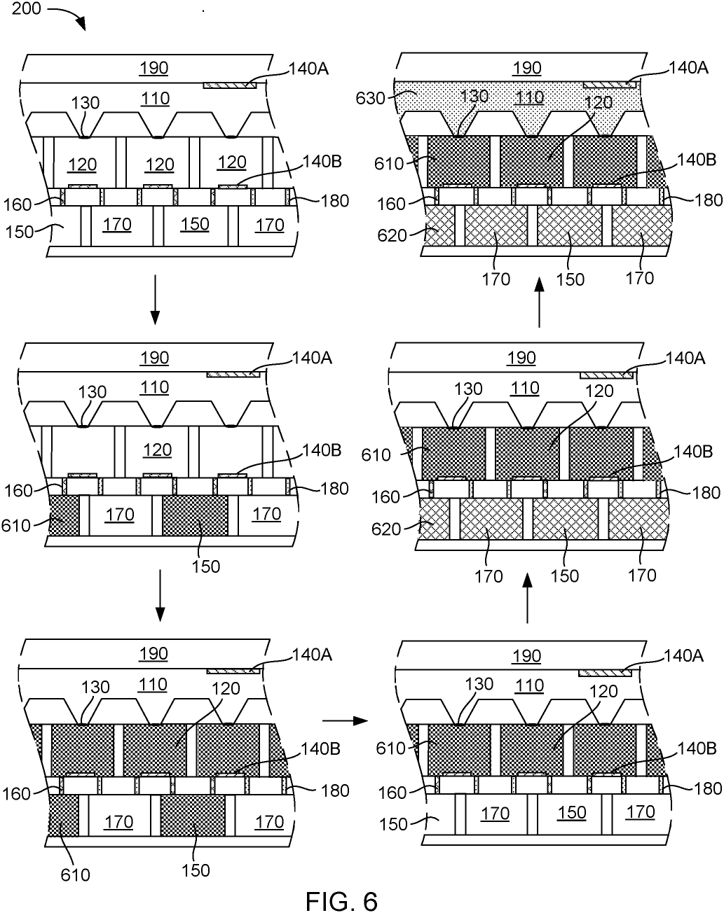

In another example, as illustrated in FIG. 6, devices, systems, and methods are further illustrated. In one example, the device and/or system components in FIG. 6 can be used to carry out the method using the microfluidic array 200. For example, the method can include loading electrolytic fluid 610 into a discrete electrolyte chamber 120. The loading can occur by loading the electrolytic fluid through an inlet channel 150 and into the discrete electrolyte chamber via an inlet port 160. In some examples, the electrolytic fluid can be wicked into the discrete electrolyte chamber. In other examples, the electrolytic fluid can be assisted by vacuum pressure into the discrete electrolyte chamber. Following loading of the electrolytic fluid into the discrete electrolyte chamber, the inlet channel can be filled with air while the outlet ports remain blocked, thereby flushing any electrolytic fluid from the inlet channel while leaving the electrolytic fluid in the discrete electrolyte channel. The inlet channel and the outlet channel can then be, or alternatively be, filled with a non-polar fluid 620. Following this, a sample electrolytic fluid can be loaded into the common electrolyte chamber. Thus, air, non-polar fluid, positive and/or negative pressure, etc., can be used in various combinations, or separately, to provide blocking for loading, filling, evacuating, etc., the individual discrete electrolyte chambers, for example.

In some examples, the microfluidic nanopore sensing device can be a microfluidic array as illustrated in FIGS. 2 and 6. The method can further include loading a second discrete electrolyte chamber with an electrolytic fluid. The loading of the second discrete electrolyte chamber can occur in concert with the initial loading of the electrolytic fluid or can occur via multiple loading steps. The loading may be dependent on an arrangement of the inlet channel of the microfluidic nanopore sensing device.

In yet another example, the method can further include nanopore sequencing of a biological sample. Nanopore sequencing can include applying a voltage to a first electrode and applying an opposite voltage to a second electrode. Upon application of the voltages, a current can be created in the microfluidic nanopore sensing device and a constant current can be generated across the nanopore opening. The current can cause the biological sample and ions from the electrolytic fluid to be driven through the nanopore opening from the first area to another area. The transport can occur from the common electrolyte chamber to the discrete electrolyte chamber or can occur from the discrete electrolyte chamber to the common electrolyte chamber. The direction of flow can initially depend on a location of the sample electrolytic fluid; however, directionality can be related to electrode polarity. Reversing a flow of the biological sample through the nanopore can occur by reversing the polarity. For example, when the sample electrolytic fluid is loaded into a common electrolyte chamber the biological sample can pass through the nanopore opening and into the discrete electrolyte chamber. As the biological sample passes through the nanopore opening, the current changes. The current change can correspond with a structure of the biological sample which can then be correlated to components of a biological sample, thereby indicating a structure of the biological sample passing therethrough. If a polarity of the electrodes is reversed, then the biological sample can repass through the nanopore opening from the discrete electrolyte chamber to the common electrolyte chamber. In an example, the biological sample can be a nucleic acid, the nanopore opening can have a diameter from 0.5 nm to 2.5 nm, and the method can further include sequencing the nucleic acid.

Over time ions in both the sample electrolytic fluid and the electrolytic fluid can be depleted. Eventually, these electrolytic fluids may be replaced to ensure proper performance of the microfluidic nanopore sensing device. The inlet channel and inlet port of the microfluidic nanopore sensing devices and systems herein can permit loading of an electrolytic fluid into the discrete electrolyte chamber. The outlet channel and outlet port of the microfluidic nanopore sensing devices and systems herein can permit flushing of an electrolytic fluid therefrom. The ability to replace an electrolytic fluid in the discrete electrolyte chamber can lengthen an operational lifespan of the microfluidic nanopore sensing device while ensuring that the nanopore opening is not damaged during loading.

It is noted that, as used in this specification and the appended claims, the singular forms “a,” “an,” and “the” include plural referents unless the content clearly dictates otherwise.

As used herein, a plurality of items, structural elements, compositional elements, and/or materials may be presented in a common list for convenience. However, these lists should be construed as though members of the list are individually identified as separate and unique members. Thus, no individual member of such list should be construed as a de facto equivalent of any other member of the same list solely based on their presentation in a common group without indications to the contrary.

Concentrations, dimensions, amounts, and other numerical data may be presented herein in a range format. It is to be understood that such range format is used merely for convenience and brevity and should be interpreted flexibly to include not only the numerical values explicitly recited as the limits of the range, but also to include all the individual numerical values or sub-ranges encompassed within that range as if numerical values and sub-ranges are explicitly recited. For example, a weight ratio range of

1 wt% to 20 wt% should be interpreted to include not only the explicitly recited limits of 1 wt% and 20 wt%, but also to include individual weights such as 2 wt%, 11 wt%, 14 wt%, and sub-ranges such as 10 wt% to 20 wt%, 5 wt% to 15 wt%, etc.

What is claimed is:

1. A microfluidic nanopore sensing device, comprising:
a common electrolyte chamber;
a discrete electrolyte chamber separated from the common electrolyte chamber by a nanopore opening there between;
an electrical circuit including multiple electrodes, wherein the common electrolyte chamber is electrically associated with a first electrode to provide a first polarity and the discrete electrolyte chamber is electrically associated with a second electrode to provide a second polarity that is opposite the first polarity;
an inlet channel fluidly coupled to the discrete electrolyte chamber via an inlet port, wherein the inlet channel is further fluidly coupled to a second discrete electrolyte chamber via a second inlet port; and
an outlet channel separated from the inlet channel, the outlet channel fluidly coupled to the discrete electrolyte chamber by an outlet port.

2. The microfluidic nanopore sensing device of claim 1, wherein the outlet channel is further fluidly coupled to the second discrete electrolyte chamber via a second outlet port.

3. The microfluidic nanopore sensing device of claim 1, wherein the nanopore opening is defined by an inorganic membrane.

4. The microfluidic nanopore sensing device of claim 1, wherein the common electrolyte chamber further includes an electrolyte inlet and an electrolyte outlet.

5. The microfluidic nanopore sensing device of claim 1, wherein the microfluidic nanopore sensing device is part of a microfluidic array including a series of discrete electrolyte chambers individually separated from the common electrolyte chamber by individual nanopore openings, wherein the discrete electrolyte chambers individually include a corresponding series of second electrodes to provide the second polarity.

6. The microfluidic nanopore sensing device of claim 5, wherein individual discrete electrolyte chambers are independently fluidly coupled to its own input channel via a corresponding inlet port, and its own outlet channel via a corresponding outlet port.

7. The microfluidic nanopore sensing device of claim 6, wherein the individual discrete electrolyte chambers are assembled in parallel so that the inlet channel is fluidly coupled to an inlet port of the individual discrete electrolyte chamber and the outlet channel is fluidly coupled to an outlet port of the individual discrete electrolyte chamber.

8. The microfluidic nanopore sensing device of claim 1, further comprising a first loading opening to load electrolytic fluid into the common electrolyte chamber, and a second loading opening to load electrolytic fluid into the discrete electrolyte chamber through the inlet channel and inlet port.

9. A microfluidic system, comprising:
a microfluidic nanopore sensing device including:
a common electrolyte chamber,
a discrete electrolyte chamber separated from the common electrolyte chamber by a nanopore opening there between,
an electrical circuit including multiple electrodes, wherein the common electrolyte chamber is electrically associated with a first electrode to provide a first polarity and the discrete electrolyte chamber is electrically associated with a second electrode to provide a second polarity that is opposite the first polarity,
an inlet channel fluidly coupled to the discrete electrolyte chamber via an inlet port, and
an outlet channel separated from the inlet channel, the outlet channel fluidly coupled to the discrete electrolyte chamber by an outlet port; and
a non-polar fluid contained within or loadable within the inlet channel, the outlet channel, or both.

10. The system of claim 9, further comprising an electrolytic fluid, wherein the electrolytic fluid is selected from potassium chloride, silver chloride, sodium chloride, lithium chloride, magnesium chloride, calcium chloride, potassium phosphate, sodium phosphate, lithium phosphate, magnesium phosphate, calcium phosphate, potassium carbonate, calcium carbonate, sodium carbonate, lithium chloride, magnesium carbonate, sulfuric acid, potassium hydroxide, or a combination thereof.

11. A method of using a microfluidic nanopore sensing device, comprising:
loading a sample electrolytic fluid including an electrolytic fluid and a biological sample into a common electrolyte chamber;
loading electrolytic fluid into a discrete electrolyte chamber, wherein the discrete electrolyte chamber is separated from the common electrolyte chamber by a nanopore opening there between, and wherein loading occurs by passing the electrolytic fluid through an inlet channel and into the discrete electrolyte chamber via an inlet port;
flushing the inlet channel with air, a non-polar fluid or sequentially air and then non-polar fluid; and
venting the electrolytic fluid from the discrete electrolyte chamber through the outlet port and into an outlet channel.

12. The method of claim 11, wherein the biological sample is a nucleic acid, the nanopore opening has a diameter from 0.5 nm to 2.5 nm, and the method further includes sequencing the nucleic acid.

13. The method of claim 11, wherein the nanopore separating the discrete electrolyte chamber from the common electrolyte chamber is formed after loading the sample electrolytic fluid, after loading the electrolytic fluid into the discrete electrolyte chamber, and after flushing the inlet channel with the air, the non-polar fluid, or the combination thereof.

14. The method of claim 11, loading a second discrete electrolyte chamber with an electrolytic fluid.

15. The method of claim 11, wherein the microfluidic nanopore sensing device further comprises an electrical circuit including multiple electrodes, wherein the common electrode chamber is fluidly coupled to a first electrode to provide a first polarity and the discrete electrolyte chamber is fluidly coupled to a second electrode to provide a second polarity that is opposite the first polarity.
